(19) United States
(12) Patent Application Publication
(10) Pub. No.: US 2013/0090515 A1
(43) Pub. Date: Apr. 11, 2013

(54) Therapeutic Furniture Covering System Using Magnets

(71) Applicant: Alice Chang, Los Angeles, CA (US)

(72) Inventor: Alice Chang, Los Angeles, CA (US)

(21) Appl. No.: 13/459,374

(22) Filed: Apr. 30, 2012

Related U.S. Application Data
(60) Provisional application No. 61/544,811, filed on Oct. 7, 2011.

(51) Int. Cl. A61N 2/00

(52) U.S. Cl. 600/9

(57) ABSTRACT

A therapeutic furniture covering system using magnets for increasing the convenience of conducting therapy treatment in an individual's home. The furniture assembly houses embedded magnets used in the improvement of an individual's overall health: improvements in circulation, reduction of pain, and relief of osteoarthritis. While a user may rest, and simultaneously treat, an individual body member, whole-body therapy may be achieved by an individual resting his or her entire body on the furniture item within close proximity of the static magnetic field.

### CROSS-REFERENCE TO RELATED APPLICATION

The present application is related to and claims priority from prior provisional application Ser. No. 61/544,811, filed Oct. 7, 2011 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of furniture coverings and more specifically relates to therapeutic furniture covering system using magnets.

2. Description of the Related Art

Furniture pieces designed to allow an individual to sit, recline, or otherwise rest are customized by many individuals in modern society according to the individual's needs and preferences. Many of these furniture pieces are limited in function to offering generally pleasing aesthetics and basic ergonomic comfort. In some cases, the function of these furniture pieces extends to providing an individual specialized support, such as that of a hospital bed, wheelchair, and the like. As more health and wellness treatments are developed and prescribed, individuals suffering from varying ailments seek treatment options that may be conducted from the privacy and comfort of their individual homes. Often, individuals pursuing home treatment options must not only procure supportive furniture but therapeutic devices in addition to the furniture pieces. This may lead not only to cumbersome, mis-matched, and awkward furniture pieces gracing an individual's home but also to the reduction of valuable storage space. Also, an individual required to assemble a therapeutic system prior to using it may find a prescribed therapy too tiresome and inefficient to maintain a rigid schedule of treatment.

Various attempts have been made to solve the above-mentioned problems such as those found in U.S. Pat. Nos. 7,326,170; 7,678,041; 2007/0225780; 2008/0171954; and 2004/0247528. This prior art is representative of furniture systems. None of the above inventions and patents, taken either singly or in combination, is seen to describe the invention as claimed.

Ideally, a furniture system offering therapeutic effects for an individual should be compact, user friendly, aesthetically pleasing and, yet, would operate reliably and be manufactured at a modest expense. Thus, a need exists for a reliable therapeutic furniture covering system using magnets to provide convenient therapy treatments to improve the overall health of an individual avoid the above-mentioned problems.

### BRIEF SUMMARY OF THE INVENTION

In view of the foregoing disadvantages inherent in the known furniture system art, the present invention provides a novel therapeutic furniture covering system using magnets. The general purpose of the present invention, which will be described subsequently in greater detail is to provide a stylish and convenient means of conducting therapy treatment in an individual's home.

Therapeutic furniture covering system using magnets is designed to improve circulation, reduce pain, and provide other health benefits through the use of magnets. Many individuals in modern society utilize magnets to aid in improving circulation and in reducing pain in afflicted body members. In the present invention, magnets are preferably embedded into furniture coverings so that a user may reap therapeutic effects while expending minimal effort. This concept may be applied to pieces of furniture such as sofas, mattresses, recliners, loveseats, and more. In addition to embedded magnets, therapeutic furniture may be made using natural fabrics, such as leather to assist in improving the overall health of a user.

A therapeutic furniture covering system using magnets for conducting therapy treatment in a user's home is disclosed herein preferably comprising a furniture assembly. A furniture item of this furniture assembly is preferably covered by an outer surface, ideally a fabric covering, which encases an inner volume. At least one magnet is preferably ensconced in this inner volume. Ideally supported by a frame, the furniture item may be used as a resting place for a user. In preferred embodiments, the furniture item comprises a sofa.

To improve the overall health of a user, the furniture item may house at least one magnet. A static magnetic field may be provided by at least one such embedded magnet, allowing a user to reap the beneficial health effects of the magnetic field when the user rests on the furniture assembly in close proximity to the magnetic field. Several potential health benefits of such magnet therapy include: improving circulation, reducing pain, and treating osteoarthritis in a body of an individual. While a user may rest, and simultaneously treat, an individual body member, whole-body therapy may be achieved by an individual resting his or her entire body on the furniture item within close proximity of the static magnetic field.

Any magnets embedded within the furniture item are preferably independent of a power source; however, the possibility exists for embedded magnets to be in communication with a power supply. While the presence of at least one embedded magnet is essential to providing the therapeutic effects of the furniture assembly, any magnet encased in the furniture item need not diminish the ergonomic comfort or aesthetic effect of the furniture assembly. In preferred embodiments, each magnet embedded in the furniture item may be about 3 cm in diameter and about 3 mm in depth. Each magnet may be embedded about 10 cm apart from one another at a depth of about 2 cm beneath the outer surface of the furniture item.

A method of using a therapeutic furniture covering system using magnets is also described herein preferably comprising the steps of: resting upon at least one furniture item; positioning at least one afflicted body member within close proximity to at least one embedded magnet; adjusting positioning of the afflicted body member as needed to maximize comfort and exposure to the magnetic field; and maintaining optimal body member positioning during an exposed period. The method preferably further comprises the step of removing the afflicted body member from close proximity to at least one embedded magnet and re-exposing the afflicted body member at a future time.

The present invention holds significant improvements and serves as a therapeutic furniture covering system using magnets. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, therapeutic furniture covering system using magnets, constructed and operative according to the teachings of the present invention.

FIGS. 1A-1C show perspective views illustrating a therapeutic furniture covering system using magnets in an in-use condition according to an embodiment of the present invention.

FIGS. 2A and 2B are perspective views illustrating the therapeutic furniture covering system using magnets as a sofa and a loveseat, respectively, with embedded magnets according to an embodiment of the present invention of FIGS. 1A-1C.

FIGS. 3A-3C are perspective views illustrating the therapeutic furniture covering system using magnets as a chair, an office chair, and a recliner, respectively, with embedded magnets according to an embodiment of the present invention of FIGS. 1A-1C.

FIGS. 4A and 4B are perspective views illustrating the therapeutic furniture covering system using magnets as a bed and a pillow with a pillow case, respectively, with embedded magnets according to an embodiment of the present invention of FIGS. 1A-1C.

FIG. 5 is a flowchart illustrating a method of use of the therapeutic furniture covering system using magnets according to an embodiment of the present invention of FIGS. 1A-4B.

The various embodiments of the present invention will hereinafter be described in conjunction with the appended drawings, wherein like designations denote like elements.

### DETAILED DESCRIPTION

As discussed above, embodiments of the present invention relate to a furniture covering device and more particularly to a therapeutic furniture covering system using magnets as used to improve the convenience of conducting therapy treatment in an individual's home.

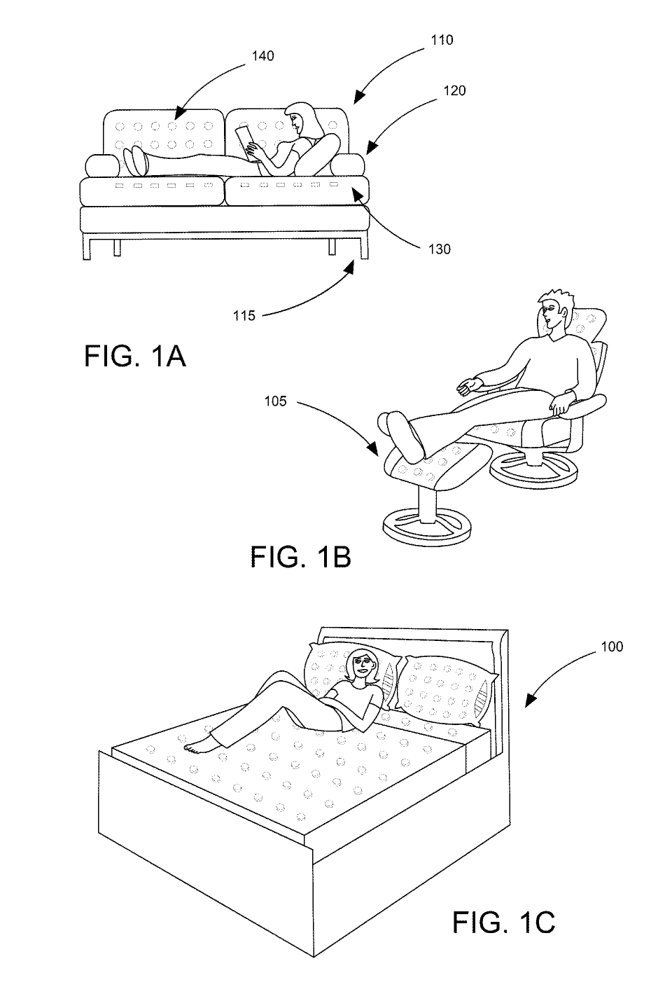

Referring to the drawings by numerals of reference there is shown in FIGS. 1A-1C, perspective views illustrating therapeutic furniture covering system using magnets 100 in an in-use condition according to an embodiment of the present invention.

Generally speaking, therapeutic furniture covering system using magnets 100 provides individuals with at least one furniture item 110 with at least one embedded magnet 140 used to improve a user's health. Any individual resting on furniture item 110 may benefit from improved circulation and reduction of pain within his or her body when resting on furniture assembly 105 and in close proximity of at least one embedded magnet 140. This concept may be applied to a variety of furniture item(s) 110, including sofa 149; loveseat 151; chair 153; recliner 155; mattress 159; and pillow 161, with or without pillow case 163. Embedded magnet(s) 140 are preferably disc-shaped. In the preferred embodiment, the remainder of the furniture assembly 105 may be made using rubber foam, leather, and the like. Furniture item(s) 110 may be manufactured in a wide variety of colors and designs. The exact specifications may vary according to user preferences.

Therapeutic furniture covering system using magnets 100 preferably comprises furniture assembly 105. Furniture assembly 105 in the preferred embodiment may comprise at least one furniture item 110 and at least one embedded magnet 140. Furniture item 110 preferably comprises: outer surface 120; inner volume 130; and frame 115. At least one embedded magnet 140 may be located within inner volume 130 of furniture item 110. In the present embodiment, outer surface 120 preferably covers furniture item 110 such that inner volume 130 may be encased by outer surface 120. Within the preferred embodiment, outer surface 120 may be comprised of leather covering 125. In combination with at least one embedded magnet 140, leather covering 125 may assist in improving the overall health of individual users. Alternatively, outer surface 120 may comprise fabric covering 123 or any suitable material for encasing inner volume 130 such as plastic, wood, or the like. Inner volume 130 may comprise an air- or material-filled void.

Embedded magnet 140 may be disc-shaped 3950 Gauss ceramic North facing permanent magnets made by mixing ceramic clay and iron powder. It should be noted that other Gauss levels may be used as per a desired strength of embedded magnet 140. Upon reading this specification, it should be appreciated that, under appropriate circumstances, considering such issues as user preferences, design preference, structural requirements, marketing preferences, cost, available materials, technological advances, etc., other magnetic arrangements such as, for example, Neodymium Iron Boron, Samarium Cobalt, Alnico etc., may be sufficient.

Materials used to fill inner volume 130 may include natural or synthetic fibrous material (cotton, polyester, etc.); Styrofoam® blocks, beads, or forms; or the like. Frame 115 preferably supports furniture item 110 in such a manner that at least one individual may rest upon furniture assembly 105. Frame 115 may be constructed using various materials such as wood, metal, plastic, or the like. Upon reading this specification, it should be appreciated that, under appropriate circumstances, considering such issues as user preferences, design preference, structural requirements, marketing preferences, cost, available materials, technological advances, etc., other outer surface, inner volume, and frame material arrangements such as, for example, rubber foam, bamboo, etc., may be sufficient. Those with ordinary skill in the art will now appreciate that upon reading this specification and by their understanding the art of furniture construction as described herein, methods of constructing furniture assemblies will be understood by those knowledgeable in such art.

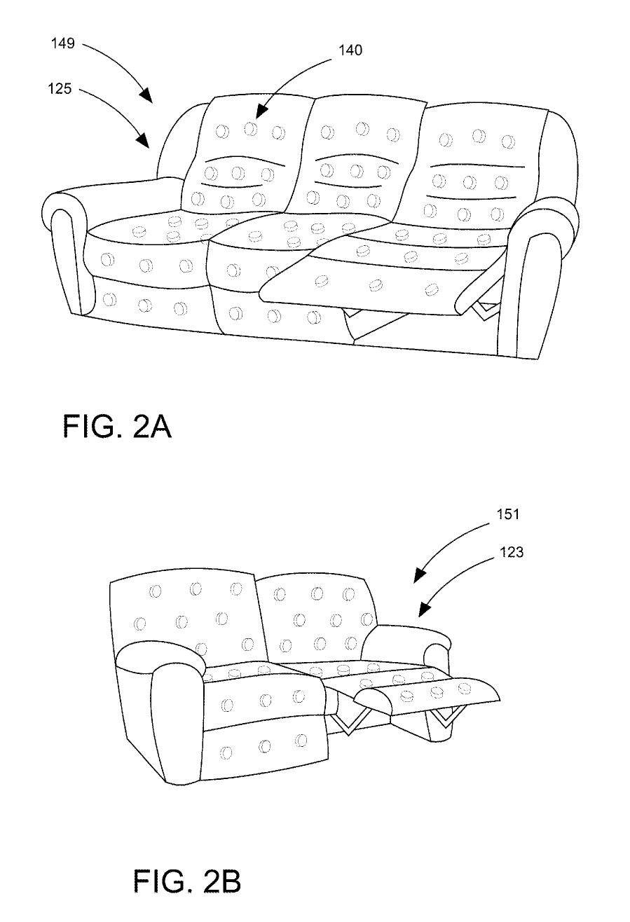

Referring now to FIGS. 2A and 2B, perspective views illustrating therapeutic furniture covering system using magnets 100 as sofa 149 and loveseat 151, respectively, with embedded magnet(s) 140 according to an embodiment of the present invention of FIGS. 1A-1C.

The preferred embodiment of therapeutic furniture covering system using magnets 100 may comprise furniture item 110. Furniture item 110 preferably comprises sofa 149. Based on user needs and preferences, the present invention may comprise loveseat 151. Varying styles and sizes may be manufactured to accommodate individual user preferences. Encased within inner volume 130 of furniture item 110 is at least one embedded magnet 140. Embedded magnet(s) 140 of the preferred embodiment may take the shape of a disk and may measure about 3 cm in diameter and about 3 mm in depth. Other manifestations of the invention may comprise embedded magnet(s) 140 of alternate shapes and sizes. To provide uniform distribution throughout furniture item 110, embedded magnet(s) 140 are preferably spaced approximately 10 cm apart from one another at a depth of about 2 cm beneath outer surface 120 of furniture item 110. Other embodiments may provide for alternate spacing and placement of embedded magnet(s) 140 within or upon furniture item 110.

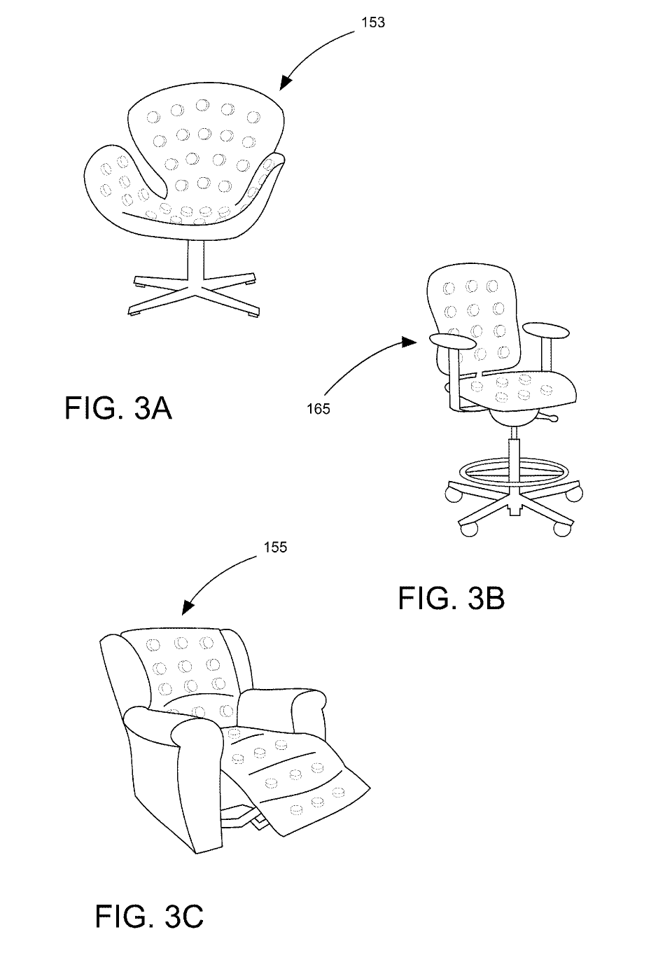

Referring now to FIGS. 3A-3C, are perspective views illustrating therapeutic furniture covering system using magnets 100 as chair 153, office chair 165, and recliner 155, respectively, with embedded magnet(s) 140 according to an embodiment of the present invention of FIGS. 1A-1C.

In addition to multiple-seat furniture item(s) 110 of the present invention, single-seat furniture item(s) 110 may be manufactured. Furniture item 110 of the present embodiment may comprise chair 153, office chair 165, and recliner 155. Whether multiple seat or single seat arrangements are provided by furniture item 110 of the present embodiment, embedded magnet(s) 140 encased in furniture item 110 provide static magnetic fields. These static magnetic fields assist an individual resting on furniture assembly 105 in improving blood flow and treating osteoarthritis within a body, while offering other potential health benefits. Those with ordinary skill in the art will now appreciate that upon reading this specification and by their understanding the art of magnet therapy as described herein, methods of improving health using magnet therapy will be understood by those knowledgeable in such art.

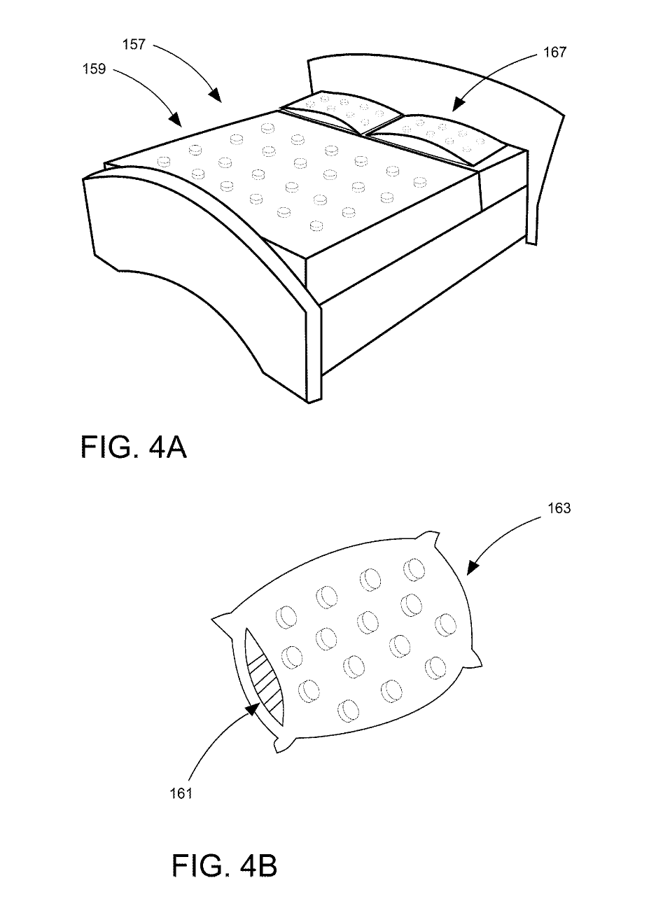

Referring now to FIGS. 4A and 4B, perspective views illustrating therapeutic furniture covering system using magnets 100 as bed 157 and pillow 161 with pillow case 163, respectively, with embedded magnet(s) 140 according to an embodiment of the present invention of FIGS. 1A-1C.

Therapeutic furniture covering system using magnets 100 of the present embodiment may comprise bed 157, including mattress 159. Mattress 159 preferably encloses at least one embedded magnet 140. Similarly, pillow(s) 161 may encase at least one embedded magnet 140, comprising bedding assembly 167. Pillow(s) 161 may be covered by pillow case(s) 163 or may rest uncovered on furniture item 110 such as bed 157, sofa 149, loveseat 151, chair 153, or the like. Pillow(s) 161 may also be used independent of other furniture item(s) 110 and placed on floors, tables, or other available surfaces.

As embedded magnet(s) 140 are diverse in shape, size, and strength, so are they versatile in function. Therapeutic furniture covering system using magnets 100 preferably comprises embedded magnet(s) 140 independent of a power source. In other embodiments, therapeutic furniture covering system using magnets 100 may comprise embedded magnet(s) 140 in communication with a power source. Those with ordinary skill in the art will now appreciate that upon reading this specification and by their understanding the art of magnets, magnetism, and magnetic fields as described herein, methods of supplying magnetic fields will be understood by those knowledgeable in such art.

Therapeutic furniture covering system using magnets 100 may be sold as kit 240 comprising the following parts: at least one furniture item 110 at least one embedded magnet 140; and at least one set of user instructions. Therapeutic furniture covering system using magnets 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

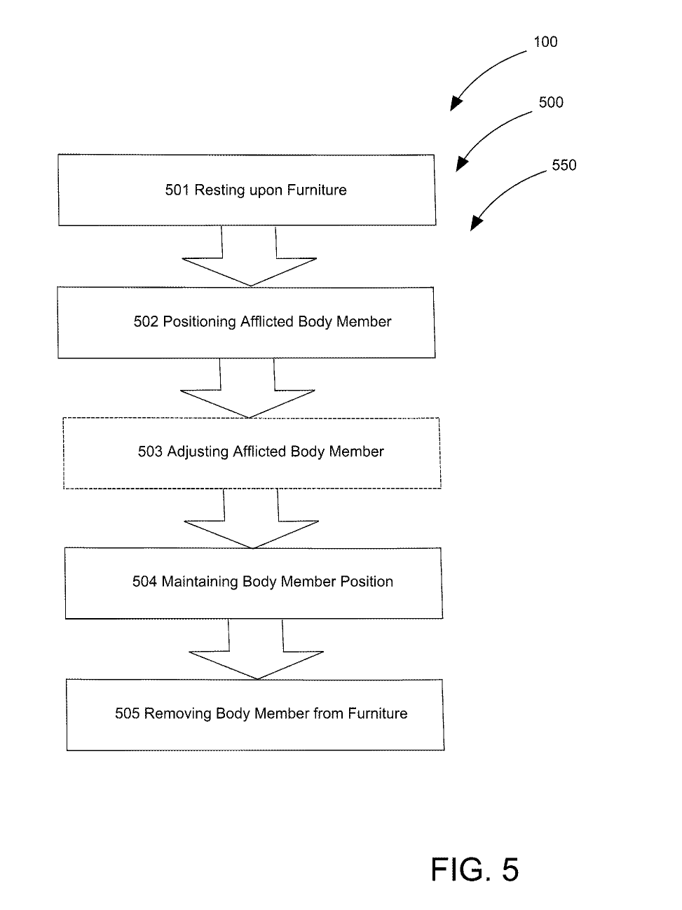

Referring now to FIG. 5, flowchart 550 illustrating method of use 500 of the therapeutic furniture covering system using magnets 100 according to an embodiment of the present invention of FIGS. 1A-4B.

A method of using (at least hereby enabling method of use 500) a therapeutic furniture covering system using magnets 100 preferably comprises the steps of: step one 501 resting upon at least one furniture item 110; step two 502 positioning at least one afflicted body member within close proximity to at least one embedded magnet 140; step three 503 adjusting positioning of afflicted body member as needed to maximize comfort and exposure to magnetic field; and step four 504 maintaining optimal body member positioning during an exposed period. The method of use 500 preferably further comprises the step of: step five 505 removing afflicted body member from close proximity to at least one embedded magnet 140 and re-exposing afflicted body member at a future time.

It should be noted that step three 503 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A therapeutic furniture covering system using magnets comprising:
a furniture assembly including;
at least one furniture item having;
an outer surface;
an inner volume; and
a frame; and
at least one embedded magnet located within said inner volume of said furniture item;
wherein said outer surface covers said furniture item and encases said inner volume, said furniture item supported by said frame such that at least one individual is able to rest thereon; and
wherein said therapeutic furniture covering system using magnets is useful for improving circulation and in reducing pain in a body of said individual as said individual, when resting on said furniture assembly, is located within a proximity of said embedded magnet(s).

2. The therapeutic furniture covering system using magnets of claim 1 wherein said furniture item comprises a bed, a mattress of said bed comprising said embedded magnet(s).

3. The therapeutic furniture covering system using magnets of claim 2 wherein said embedded magnet(s) are enclosed within pillows, said pillows covered by pillow cases.

4. The therapeutic furniture covering system using magnets of claim 3 wherein said pillows comprising said embedded magnet(s) comprise a bedding assembly.

5. The therapeutic furniture covering system using magnets of claim 1 wherein said furniture item comprises a chair.

6. The therapeutic furniture covering system using magnets of claim 5 wherein said chair comprises a recliner.

7. The therapeutic furniture covering system using magnets of claim 1 wherein said furniture item comprises a sofa.

8. The therapeutic furniture covering system using magnets of claim 1 wherein said sofa comprises a loveseat.

9. The therapeutic furniture covering system using magnets of claim 1 wherein said outer surface comprises a leather covering.

10. The therapeutic furniture covering system using magnets of claim 7 wherein said leather covering in combination with said embedded magnet(s) assist in improving the overall health of said individual(s).

11. The therapeutic furniture covering system using magnets of claim 1 wherein said outer surface comprises a fabric covering.

12. The therapeutic furniture covering system using magnets of claim 1 wherein said embedded magnet(s) provide static magnetic fields.

13. The therapeutic furniture covering system using magnets of claim 12 wherein said static magnetic fields improve blood flow in said individual resting on said furniture item.

14. The therapeutic furniture covering system using magnets of claim 1 wherein said embedded magnet(s) are located in said inner volume independent of a power source.

15. The therapeutic furniture covering system using magnets of claim 1 wherein said embedded magnet(s) are about 3 cm in diameter and about 3 mm in depth, said embedded magnet(s) imbedded about 10 cm apart from one another and at a depth of about 2 cm beneath said outer surface of said furniture item.

16. The therapeutic furniture covering system using magnets of claim 1 wherein said embedded magnet(s) are in communication with a power source.

17. The therapeutic furniture covering system using magnets of claim 1 wherein said therapeutic furniture covering system using magnets is useful in treating osteoarthritis.

18. A therapeutic furniture covering system using magnets comprising
a furniture assembly including;
a furniture item having;
an outer surface;
an inner volume; and
a frame; and
at least one embedded magnet located within said inner volume of said furniture item;
wherein said outer surface covers said furniture item and encases said inner volume, said furniture item supported by said frame such that at least one individual is able to rest thereon;
wherein said furniture item of said therapeutic furniture covering system using said embedded magnets comprises a sofa;
wherein said embedded magnet(s) are about 3 cm in diameter and about 3 mm in depth, said embedded magnet(s) imbedded about 10 cm apart from one another and at a depth of about 2 cm beneath said outer surface of said furniture item;
wherein said embedded magnet(s) are independent of a power source;
wherein said outer surface comprises a fabric covering; and
wherein said therapeutic furniture covering system using magnets is useful for improving circulation, reducing pain, and treating osteoarthritis in a body of said individual as said individual, when resting on said furniture assembly, is located within a proximity of a static magnetic field provided by said embedded magnet(s) which assists in improving the overall health of said individual(s).

19. A method of using a therapeutic furniture covering system using magnets comprising the steps of:
resting upon at least one furniture item;
positioning at least one afflicted body member within close proximity to at least one embedded magnet;
adjusting positioning of said afflicted body member as needed to maximize comfort and exposure to magnetic field; and
maintaining optimal body member positioning during an exposed period.

20. The method of claim 19 further comprising the step of removing said afflicted body member from said close proximity to said at least one embedded magnet and re-exposing said afflicted body member at a future time.
